(19) United States
(12) Patent Application Publication
(10) Pub. No.: US 2011/0034940 A1
(43) Pub. Date: Feb. 10, 2011

(54) SYSTEMS AND METHODS FOR GUIDING A MEDICAL INSTRUMENT

(76) Inventor: Troy D. Payner, Indianapolis, IN (US)

Correspondence Address: SCHWEGMAN, LUNDBERG & WOESSNER, P.A., P.O. BOX 2938, MINNEAPOLIS, MN, 55402, US

(21) Appl. No.: 12/905,622

(22) Filed: Oct. 15, 2010

Related U.S. Application Data
(63) Continuation of application No. PCT/US2009/062566, filed on Oct. 29, 2009.
(60) Provisional application No. 61/109,662, filed on Oct. 30, 2008.

(51) Int. Cl. A61B 19/00

(52) U.S. Cl. 606/130

(57) ABSTRACT

This document discusses, among other things, a system for guiding a medical instrument within a body of a subject. The system includes a generic anatomical representation of at least a portion of the subject, a processing device, and a display device communicatively coupled to the processing device. The processing device is configured to determine a position of the medical instrument relative to the generic anatomical representation based on a position of the medical instrument relative to the subject. The display device configured to display the generic anatomical representation and a representation of the position of the medical instrument relative to the generic anatomical representation.

### CLAIM OF PRIORITY

This patent application is a continuation under 35 U.S.C. 111(a) of International Application No. PCT/US2009/062566, filed on Oct. 29, 2009 and published on May 27, 2010 as WO 2010/059375, which claims the benefit of priority, under 35 U.S.C. Section 119(e), to U.S. Provisional Patent Application Ser. No. 61/109,662, filed on Oct. 30, 2008, which applications and publication are incorporated herein by reference in their entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the products, processes, and data as described below and in the tables that form a part of this document: Copyright 2008, Dr. Troy Payner. All Rights Reserved.

### BACKGROUND

Physicians place ventricular catheters in patients' brains to treat conditions such as elevated intracranial pressure or hydrocephalus. Catheter placement in a cerebral ventricle is a risky and sometimes difficult procedure, with false trajectories resulting in increased morbidity. While stereotactic guidance mechanisms are currently available, these mechanisms are costly and time consuming, and they require the patient to go to the operating room for the procedure. What is needed is a faster, easier, and less expensive method for guided cerebral catheter placement, such as one that can be performed in an intensive care unit.

Typical catheter positioning systems require the use of expensive and complicated imaging systems to provide images of the cerebrum of the subject. These imaging systems may increase the length of time required for a procedure due to a large time requirement for set-up of the subject and the imaging system. These imaging systems may also increase the cost for some otherwise relatively simple procedures.

### OVERVIEW

This document discusses, among other things, a system for guiding a medical instrument within a body of a subject. The system includes a generic anatomical representation of at least a portion of the subject, a processing device, and a display device communicatively coupled to the processing device. The processing device is configured to determine a position of the medical instrument relative to the generic anatomical representation based on a position of the medical instrument relative to the subject. The display device configured to display the generic anatomical representation and a representation of the position of the medical instrument relative to the generic anatomical representation.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals can describe substantially similar components throughout the several views. Like numerals having different letter suffixes can represent different instances of substantially similar components. The drawings illustrate generally, by way of example, but not by way of limitation, various embodiments discussed in the present document.

FIGS. 1A and 1B are schematic diagrams illustrating generally examples of a guided catheter placement system.

FIG. 2 is a flow chart illustrating generally an example of a technique for guiding placement of a catheter in a subject's cerebral ventricle.

FIGS. 3A, 3B, and 3C are schematic diagrams illustrating generally an example of a technique for positioning magnetic sensors to be used in a guided catheter placement system.

FIG. 4 is a flow chart illustrating generally an example of a method for guiding a medical instrument with a body of a subject.

FIG. 5 illustrates generally one example of a system for guiding a medical instrument within a body of a subject.

FIG. 6 illustrates one example of a reference frame of a magnetic sensor in relation to a reference frame of an emitter.

### DETAILED DESCRIPTION

Invention embodiments described herein include, among other things, method embodiments for the guided placement of a catheter in a cerebral ventricle of a subject's brain using a magnetic tracking and imaging device. Such a device is usable by medical professionals to safely and accurately insert a catheter into a subject's cerebral ventricle. Safety and accuracy in this positioning procedure are important because false trajectories, missing the cerebral ventricle, can lead to cerebral hemorrhage and stroke. Repeatedly passing the catheter also increases the risk of infection.

The present inventor has recognized, among other things, that some medical procedures can be performed quickly and inexpensively with a guiding system that uses a plurality of magnetic sensors that sense a position and orientation of at least one emitter on a medical instrument. The present inventor has recognized that during some medical procedures an actual image or images of the subject may not need to be obtained during the procedure. Instead, the position and orientation of the guiding system are displayed relative to an average anatomical representation of the portion of the subject of interest. In particular, the present inventor has recognized that when guiding a medical instrument to a location based on a normal and predictable anatomical makeup, an average anatomical representation of a subject can be used, and an actual image of the subject may not be necessary. Accordingly, the use of potentially expensive and time consuming imaging systems can be avoided during some medical procedures.

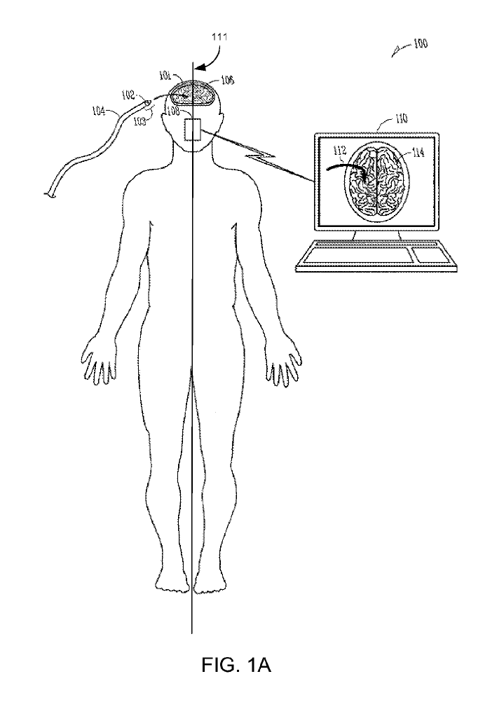

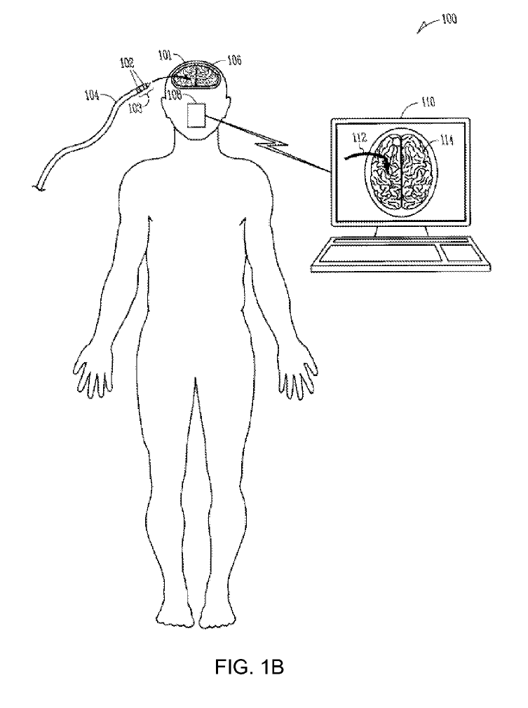

One inventive embodiment, illustrated generally and schematically at 100 in FIG. 1A includes a guided catheter placement system 100, such as for use with a human or animal subject 101. System 100 includes at least one magnetic emitter 102 coupled to a flexible catheter 104 that is of a size effective for entering a cerebral ventricle and has a firmness effective for travel through brain tissue. In this embodiment, two or more magnetic emitters 102 are embedded within the tip 103 of the catheter 104 or attached to the tip of a stylet that can be inserted into the catheter and then removed once the catheter is inserted into the ventricle. Alternatively, the magnetic emitters 102 are reversibly attachable to the catheter 104. The magnetic emitters 102 are, for some embodiments, a current-carrying coil or permanent magnet. In the example illustrated in FIG. 1B, there are two magnetic emitters 102 embedded within the tip 103 of the catheter 104.

In the preferred embodiment, the magnetic emitter(s) 102 in FIGS. 1A and 1B can generate at least one magnetic field, which, in turn, can be sensed by magnetic sensors 306 as the catheter 104 is being placed in a subject's brain 106. Furthermore, the magnetic sensors are communicatively linked to a user interface 110, displayed on a computer monitor or similar display. As the catheter 104 is inserted into the brain 106, the trajectory 112 of the magnetic emitters 102 coupled to the catheter 104 can be graphically displayed on the user interface 110. The graphic display may be represented by a grid that identifies a midline 111 (shown in FIG. 1A) as well as the distance from anterior, posterior, and bilateral external magnetic sensors. As referred to herein, the midline 111 in 2-D form is a plane (referred to as the midplane 111) that bisects a portion of the subject. In one example, the midplane 111 bisects the portion of the subject symmetrically. In certain examples, the graphic display can be configured to display a graphical representation of an average (generic) anatomy.

The trajectory 112 of the catheter 104 can be displayed in association with an image of the brain 114, such that the actual, real-time location of the magnetic emitter 102 in the brain 106 is reflected in the display on the user interface 110. Alternatively, in positioning the catheter prior to and during insertion into the brain, the trajectory can be displayed by a circular graph; holding the entry point fixed, the cone-shaped range of motion of all possible angles can be swept through a circular pattern, thus a circular graph is an ideal format for showing the current trajectory relative to the desired trajectory.

When two magnetic emitters 102 are used, such as in FIG. 1B, a line can be generated between the first and second magnetic emitters 102 on the user interface display 110. As the catheter advances into the brain 106, the path of each magnetic emitter 102 can be tracked, such that the trajectory image display 112 is two-dimensional as it is displayed on the user interface 110. In certain examples, components of the trajectory not visible on the two-dimension display 112 can also be displayed graphically. Alternatively, a trajectory may be determined, as in the embodiment of FIG. 1A, by determining the orientation of the single magnetic emitter 102.

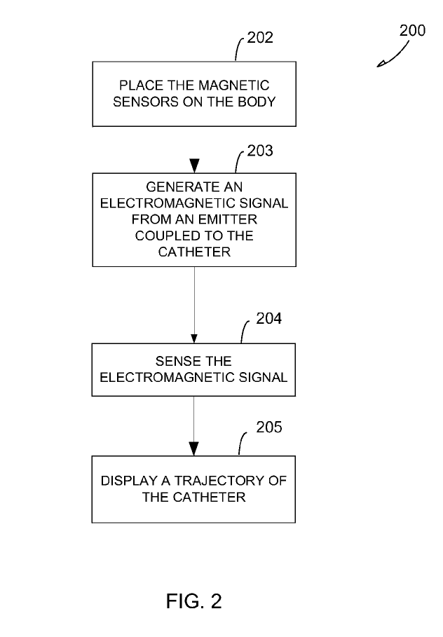

FIG. 2 illustrates one method at 200 for guiding placement of a catheter in a subject's cerebral ventricle. At 202, two or more magnetic sensors are positioned on the subject's body. As described below, the magnetic sensors provide landmarks that help to accurately track the trajectory of the catheter within the brain. Typically, an orifice, or bun hole, is drilled on the right or left side of the subject's skull in the midpupillary line by conventional techniques. After the drilling of the orifice is complete, the dura is opened, as is well known in the art, and the catheter is placed at the opening in preparation for insertion. At 203, a magnetic field is generated from a magnetic emitter, which is coupled to a catheter that is to be placed in a cerebral ventricle. At 204, the magnetic field is sensed by the magnetic sensors as the catheter is inserted into the brain. At 205, a graphic representation of the trajectory of the catheter, based on the sensed magnetic field, is displayed on a user interface. The catheter can then be advanced into the brain tissue towards the cerebral ventricle. The position of the catheter can be tracked along the path to the ventricle, as steps 203 through 205 are continuously repeated, to maintain a precise trajectory to the target.

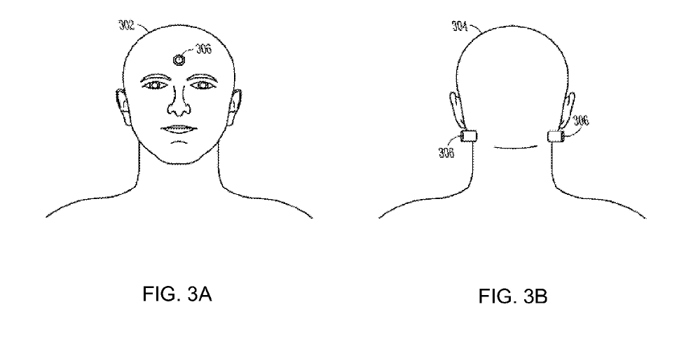

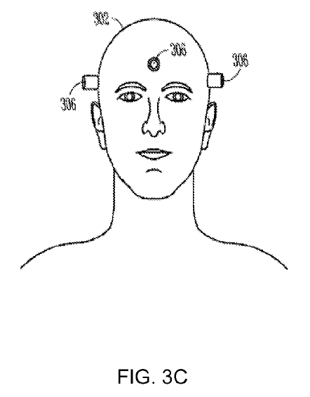

FIGS. 3A, 3B, and 3C schematically illustrate methods for positioning magnetic sensors 306 usable in a guided catheter placement system. In this embodiment, magnetic sensors 306 are attached to a subject's head 302. In one embodiment, magnetic sensors are attached to a subject's body surface by way of an adhesive backing. Using the same method of tracking as described above for the magnetic emitter 102 coupled to the tip of the catheter 104, the magnetic sensors 306 are usable to generate a graphic representation on the user interface 110 of various anatomical markers. For example, in an embodiment shown in FIG. 3A the magnetic sensor 306 is placed on the subject's forehead. As illustrated in FIG. 3B, magnetic sensors 306 can also be placed in the pre- or post-auricular surfaces of the subject's skin. FIG. 3C illustrates another possible location on the temples. Furthermore, the magnetic sensors 306 are usable to generate a graphic representation of the midline, or a line/plane representative of other anatomic features, of a subject's brain by generating a line between two or more magnetic sensors 306. By generating such landmarks, the magnetic sensors 306 help a medical professional to accurately track the trajectory of the catheter 104 within the brain.

While certain types of magnetic emitters 102 and magnetic sensors 306 are described herein, it is to be understood that various other types of magnetic emitters or magnets can be used.

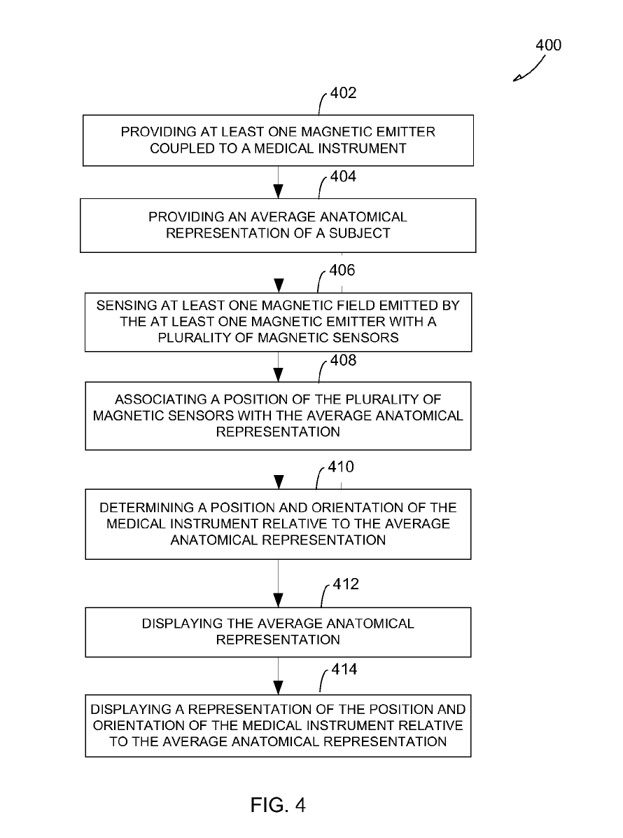

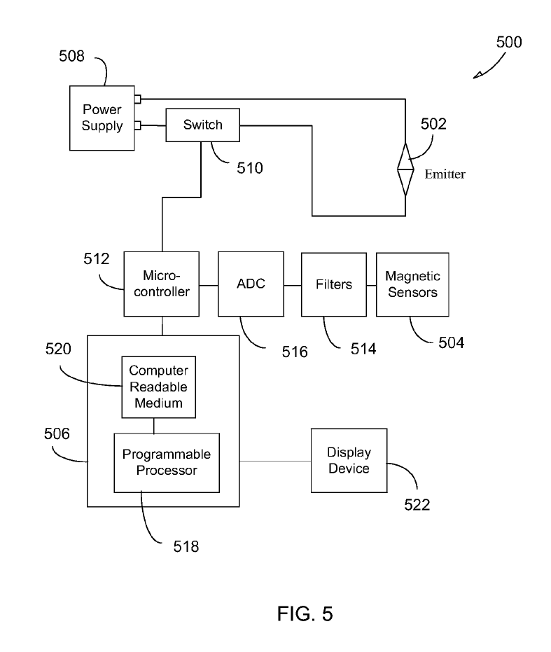

FIG. 4 illustrates one method 400 for guiding a medical instrument within a body of a subject. FIG. 4 is explained below with reference to the system shown in FIG. 5. Numbers in parenthesis (400) refer to acts of method 400.

FIG. 5 illustrates generally one example of a system 500 for guiding placement of a medical instrument within a body. System 500 includes at least one magnetic emitter 502 for generating at least one magnetic field (400), a plurality of magnetic sensors 504 for sensing the at least one magnetic field, and a processing device 506 for interfacing with a user. In some examples, the at least one magnetic emitter 502 includes one or more emitting coils 502 to generate a one or more magnetic fields. In one example, each of the plurality of magnetic sensors 504 include 3-axis magnetometers that are capable of sensing a three dimensional magnetic field. The magnetic emitter 502 and the plurality of magnetic sensors 504 operate substantially the same as the emitter 102 and the sensors 306 described above and as further described below. In some examples, the processing device includes a personal computer, a user interface device, and the like.

System 500 also includes a power supply 508 to provide power to the at least one magnetic emitter 502, and a switching device 510 to control when the at least one magnetic emitter 502 is emitting a magnetic field. System 500 also includes a microcontroller 512 to interface with the processing device 506 and the plurality of magnetic sensors 504, and control the switching device 510. In other words, the microcontroller 512 closes the switching device 510 to couple power from the power supply 508 to the at least one magnetic emitter 502. The microcontroller 512 couples power to the at least one magnetic emitter 502 when the system 500 is ready to take a measurement of the magnetic field from the at least one magnetic emitter 502. In one example, the microcontroller 512 can open the switching device 510 between measurements.

The at least one magnetic field generated by the at least one magnetic emitter 502 is sensed by the plurality of magnetic sensors 504 (406). The plurality of magnetic sensors 504 then output a signal indicative of the sensed at least one magnetic field. In one example, the signal output from the plurality of magnetic sensors 504 is filtered by a filter 514 to reduce noise, and converted into a digital signal by an analog-to-digital converter (ADC) 516. In one example, the filter 514 comprises a low-pass filter and ach output from the magnetic sensors 504 is passed through the low-pass filter in order to get rid of any AC components in the output signal.

The microcontroller 512 receives and analyzes outputs from the ADC 516 and sends information indicative of the signal to the processing device 506. In one example, the information sent from the microcontroller 512 is sent via USB to the processing device 506, where it is processed. In one example, an optical isolator is used to separate power systems of the microprocessor 512 and the at least one magnetic emitter 502, since the microprocessor 512 and the at least one magnetic emitter 502 use different power supplies and grounds.

The processing device 506 includes a programmable processor 518 and a storage device 520. Storage device 520 includes a computer readable media having software stored thereon. The software includes instructions for execution by programmable processor 518. In some examples, storage device 520 may include, but are not limited to, hard disks, removable magnetic disks, removable optical disks (e.g., compact disks and digital video disks), magnetic cassettes, memory cards or sticks, random access memories (RAMs), read only memories (ROMs), and the like. In some examples, the programmable processor 518 may include a general purpose processor, a microcontroller, or other programmable circuitry.

In one example, the software includes instructions for the programmable processor 518 to calculate a position and an orientation of the at least one magnetic emitter 502 using data obtained by the plurality of magnetic sensors 504 regarding the at least one magnetic field from the at least one magnetic emitter 502. In one example, the programmable processor 518 calculates a position and an orientation of the at least one magnetic emitter 502 relative to the plurality of magnetic sensors 504. Once the position and orientation of the at least one magnetic emitter 502 is determined relative to the plurality of magnetic sensors 506, software instructs the programmable processor 518 to correlate the position and orientation of the at least one magnetic emitter 502 with the body of the subject. In other words, the position and orientation of the at least one magnetic emitter 502 is determined with respect to the body of the subject. In one example, the position and orientation of the at least one magnetic emitter 502 are correlated with the body of the subject by associating a reference plane respective to the plurality of sensors 504 with a midplane (e.g. midplane 111) of the portion of the subject on which the plurality of magnetic sensors 504 are located. In one example, the midplane includes a plane symmetrically bisecting the portion of the subject on which the plurality of sensors 504 are located. In one example, the plurality of magnetic sensors 405 are located on a head portion of the subject, and the midplane is a plane positioned between the left and right hemispheres of the brain of the subject. The programmable processor 518, therefore, determines the position and orientation of the at least one magnetic emitter 502 with respect to the midplane of the subject based on the position and orientation of the at least one magnetic emitter 502 as determined with respect to the plurality of sensors 504.

In some examples, the plurality of magnetic sensors 504 include a first, a second, and a third magnetic sensor that are configured to be placed on the exterior of a subject. In one example, the first and second magnetic sensors are configured to be symmetrically located on opposite sides of a subject. In other words, the first and second magnetic sensors are configured to be placed on the subject such that the midplane of the subject is substantially halfway between the first and the second magnetic sensors. In one example, the first and the second magnetic sensors are placed on the left and right temples of the subject respectively (shown in FIG. 3C). Placing the first and second magnetic sensors symmetrically on the subject, enables the midplane of the subject to be associated with a hypothetical reference plane halfway between the first and second magnetic sensors 504. Accordingly, in one example, the first and second magnetic sensors are placed symmetrically on the subject and a reference plane substantially halfway between the first and second sensors is associated with the midplane of the subject. In one example, associating the reference plane with the midplane of the subject includes associating the reference plane with a bias off of the midplane of the subject. For example, a bias can be used when an image of a brain of a subject indicates that the brain of the subject has hemorrhaged and the midplane between the left and right hemispheres has shifted 2 mm to the right respective to a typical human brain. To account for the shift of the midplane, a user can input a bias of the midplane into a user interface on the processing device 506 and the software can account for the shift by associating the reference plane halfway between the first and second sensors with a plane 2 mm off of the midplane. Regardless of whether a bias in the midplane is applied, the correlation from the position and orientation relative to the reference plane of the plurality of magnetic sensors 504 to the position and orientation relative to the midplane of the subject is accomplished using mathematical algorithms as known to those skilled in the art.

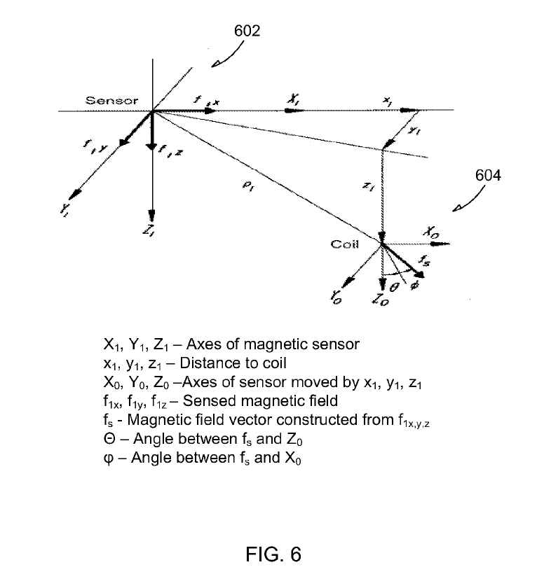

FIG. 6 illustrates one example of a reference field 602 of a magnetic sensor (e. g magnetic sensor 306 or magnetic sensor 504) in relation to a reference field 604 of an emitter (e.g. emitters 102 or emitter 502). In a first example, the position is defined as the distance away from a magnetic sensor 504 in the x, y, and z axes. In the first example, the orientation is defined as the angles theta (θ) and phi (φ) that the axes of the at least one emitter 502 differ from the axes of the magnetic sensor 504. In a second example, position and orientation are determined as the position of the magnetic sensor 504 relative to the at least one emitter 502. Both the first and second example represent essentially the same measurement, with differences in convention of the point of origin.

In one example, a third magnetic sensor of the plurality of magnetic sensors 504 is positioned on the subject. The third magnetic sensor is used to sense the at least one magnetic field from the at least one emitter 502 from a third location different than the first and second magnetic sensor. In one example, the third magnetic sensor is positioned on a forehead of the subject (shown in FIG. 3C). In some examples, the third magnetic sensor is positioned approximately on the midplane of the subject.

In one example, to determine the position and orientation of the at least one emitter 502, the at least one magnetic field of the at least one emitter 502 is modeled. The Biot-Savart law is used to describes the relationship between the magnetic field produced by a wire and current running through the wire. The Biot-Savart law can be manipulated to produce a closed form equation that relates the geometry of, and current through, a loop to the magnetic field produced at any point in space. Each magnetic emitter 502 is able to be accurately approximated by a series of loops. As an alternate form of the at least one magnetic emitter 502, a permanent magnet may also be modeled using a different form of the Biot-Savart law.

Referring back to FIG. 5, the processing device 506 is communicatively coupled to a display device 522 for interfacing with a user. In one example, the software includes instructions to configure the display device 522 (e.g. via the programmable processor 518) to display the position and orientation of the at least one emitter 502 with respect to the midplane of the subject.

In one example, the storage device 520 includes an average anatomical representation of at least a portion of the subject (404). The average anatomical representation of the brain can be associated with the plurality of magnetic sensors 502 by associating the reference plane halfway between the first and second magnetic sensors with a midplane of the anatomical representation of the brain (408). With the midplane of the anatomical representation of the brain associated with the reference plane, the position and orientation of the at least one emitter 502 can be determined relative to the anatomical representation of the brain (410). In one example, the display device 522 displays an average anatomical representation of a brain (412). The software then configures the display device 522 to display the position of the at least one emitter 502 relative to the anatomical representation of the brain (414). In certain examples, the average anatomical representation can include a compilation of a plurality of images each image acquired from a different subject. In other examples, the average anatomical representation can include a generic artistic rendering of the at least a portion of the subject.

Utilizing an average anatomical representation of a portion of the subject (e.g. the brain) enables the processing device 506 to visually display on the display device 522 the position of the at least one emitter 502 relative to the subject when the portion of the subject of interest does not vary widely from the average anatomical representation. Thus, the anatomical representation can be used without having to correlate the position of the at least one emitter 502 with an actual image of the subject. In other words, the position can be displayed without the complication and expense of a body imaging system.

In one example, a target destination for a medical device having the at least one emitter 502 thereon is displayed on the display device 522 relative to the anatomical representation and the position of the at least one emitter 502. The target destination can be known a priori and or can be input by a user. In one example, the orientation of the at least one emitter 502 is used to project a trajectory for the at least one emitter 502. For example, a straight line trajectory from the present position of the at least one emitter 502 can be projected outward based on a known association between the orientation of the at least one emitter 502 and the medical device. For example, a catheter is configured to be inserted with a tip portion leading, and when the orientation of the at least one emitter 502 is known relative to the orientation of the catheter, the direction that the tip is facing can be used to project a trajectory for the catheter. In one example, the projected trajectory is displayed relative to the target destination and/or a target trajectory. The target trajectory can be determined based on the current position of the at least one emitter 502 and the target destination, wherein the target trajectory is a line between the current position and the target destination. In one example, the target trajectory relative to the projected trajectory is displayed as a bulls eye and a dot. The bulls eye represents the target trajectory and the dot represents the present projected trajectory. By changing the orientation of the medical device (and the at least one emitter) such that the dot is centered on the bulls eye, the user can align the projected trajectory with the target trajectory.

In one example, the position and orientation of the at least one emitter 502 (and the medical instrument attached thereto) are determined prior to insertion into all or a specific portion of the body. For example, prior to insertion into a brain the position and orientation of the at least one emitter can be determined such that the proper trajectory is achieved at the beginning of the insertion and (ideally) the trajectory does not change as the medical instrument is inserted into the brain. Additionally, in an example, the position and orientation (as well as the projected trajectory) are updated on the display device 522 in order to have a real-time display of the position and orientation of the medical instrument. To update the position and orientation, the acts listed above can be repeated in a looped manner.

The examples described herein (e.g. with respect to FIG. 5) can be machine or computer-implemented at least in part. Some examples can include a computer-readable medium or machine-readable medium encoded with instructions operable to configure an electronic device (e.g. processing device 506) to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. However, the present inventor also contemplates examples in which only those elements shown and described are provided.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A system for guiding a medical instrument with respect to a subject, the system comprising:
a generic anatomical representation of at least a portion of the subject; and
a processing device configured to:
determine a position of the medical instrument relative to the generic anatomical representation based on a position of the medical instrument relative to the subject; and
a display device communicatively coupled to the processing device, the display device configured to display the generic anatomical representation and a representation of the position of the medical instrument relative to the generic anatomical representation.

2. The system of claim 1, wherein determine a position includes determine a position of the medical instrument relative to a landmark, and use a position of the landmark relative to the subject along with the position of the medical instrument relative to the landmark to determine the position of the medical instrument relative to the generic anatomical representation.

3. The system of claim 2, wherein the landmark is positioned with respect to the subject such that a relationship between the landmark and the subject is known by the system.

4. The system of claim 3, wherein the processing device is configured to:
associate the relationship between the landmark and the subject with a relationship between the landmark and the generic anatomical representation, such that the position of the landmark relative to the subject is correlated with a position of the landmark with respect to the generic anatomical representation.

5. The system of claim 4, wherein associate the relationship includes associate a reference plane with respect to the landmark with a plane with respect to the subject.

6. The system of claim 5, wherein associate the reference plane includes associate the reference plane with a midplane of at least a portion of the subject.

7. The system of claim 2, comprising:
at least one magnetic emitter coupled to the medical instrument, the magnetic emitter configured to generate at least one magnetic field; and
wherein the landmark includes a plurality of magnetic sensors configured to sense the at least one magnetic field generated by the at least one magnetic emitter.

8. The system of claim 7, comprising:
associate a position of the plurality of magnetic sensors with the generic anatomical representation; and
wherein determine a position of the medical instrument relative to the generic anatomical representation includes determine a position of the magnetic emitter relative to the plurality of magnetic sensors.

9. The system of claim 7, wherein the processing device is further configured to:
determine an orientation of the medical instrument using data from the plurality of magnetic sensors regarding the magnetic field generated by the magnetic emitter.

10. The system of claim 9, wherein the display device is configured to:
display a projected trajectory of the medical instrument using the position of the medical instrument and the orientation of the medical instrument.

11. The system of claim 10, wherein the display device is configured to:
display a target position for the medical instrument relative to the projected trajectory of the medical instrument.

12. The system of claim 1, wherein the generic anatomical representation comprises an average representation of at least a portion of a plurality of bodies corresponding to the at least a portion of the body of the subject.

13. A method for guiding a medical instrument with respect to a subject, the method comprising:
providing at least one landmark positioned relative to the subject;
providing a generic anatomical representation of at least a portion of the subject;
determining a position of the medical instrument relative to the generic anatomical representation based on a position of the medical instrument relative to the landmark; and
displaying the generic anatomical representation; and
displaying a representation of the position of the medical instrument relative to the generic anatomical representation.

14. The method of claim 13, wherein determining a position includes determining a position of the medical instrument relative to the landmark, and using a position of the landmark relative to the subject along with the position of the medical instrument relative to the landmark to determine the position of the medical instrument relative to the generic anatomical representation.

15. The method of claim 14, comprising:
associating a relationship between the landmark and the subject with a relationship between the landmark and the generic anatomical representation, such that the position of the landmark relative to the subject is correlated with a position of the landmark with respect to the generic anatomical representation.

16. The method of claim 15, wherein associating the relationship includes associating a reference plane with respect to the landmark with a plane with respect to the subject.

17. The method of claim 16, wherein associating the reference plane includes associating the reference plane with a midplane of at least a portion of the subject.

18. The method of claim 13, comprising:
providing at least one magnetic emitter coupled to the medical instrument, the magnetic emitter configured to generate at least one magnetic field; and
wherein the landmark includes a plurality of magnetic sensors configured to sense the at least one magnetic field generated by the at least one magnetic emitter.

19. The method of claim 18, comprising:
associating a position of the plurality of magnetic sensors with the generic anatomical representation; and
wherein determining a position of the medical instrument relative to the generic anatomical representation includes determining a position of the magnetic emitter relative to the plurality of magnetic sensors.

20. The method of claim 18, comprising:
determining an orientation of the medical instrument using data from the plurality of magnetic sensors regarding the magnetic field generated by the magnetic emitter.

21. The method of claim 20, comprising:
display a projected trajectory of the medical instrument using the position of the medical instrument and the orientation of the medical instrument.

22. The method of claim 21, comprising:
displaying a target position for the medical instrument relative to the projected trajectory of the medical instrument.

23. The method of claim 13, wherein the generic anatomical representation comprises an average representation of at least a portion of a plurality of bodies corresponding to the at least a portion of the body of the subject.

24. A system for guiding a medical instrument with respect to a subject, the system comprising:
a landmark positioned relative to the subject; and
a processing device configured to:
associate a reference plane relative to the landmark with a midplane of at least a portion of the subject;
determine a position of the medical instrument relative to the midplane using a position of the landmark relative to the subject along with the position of the medical instrument relative to the landmark; and
a display device communicatively coupled to the processing device, wherein the processing device is configured to direct the display device to display a representation of the position of the medical instrument relative to the midplane.

25. The system of claim 24, wherein associate a reference plane includes correlating a position of the landmark relative to the midplane with a position of the landmark with respect to the reference plane.

26. The system of claim 24, wherein the reference plane is a plane between a first and a second landmark.

27. The system of claim 24, comprising:
at least one magnetic emitter coupled to the medical instrument, the magnetic emitter configured to generate at least one magnetic field;
wherein the landmark includes a plurality of magnetic sensors configured to sense the at least one magnetic field generated by the at least one magnetic emitter; and
wherein the processing device is configured to:
wherein determine a position of the medical instrument includes determine a position of the magnetic emitter relative to the plurality of magnetic sensors.

28. The system of claim 27, wherein the processing device is further configured to:
determine an orientation of the medical instrument using data from the plurality of magnetic sensors regarding the magnetic field generated by the magnetic emitter; and
wherein the display device is configured to:
display a projected trajectory of the medical instrument using the position of the medical instrument and the orientation of the medical instrument; and
display a target position for the medical instrument relative to the projected trajectory of the medical instrument.
